(19) United States
(12) Patent Application Publication
(10) Pub. No.: US 2024/0407808 A1
(43) Pub. Date: Dec. 12, 2024

(54) MODULAR PIVOTAL BONE ANCHOR WITH BOTTOM-LOADED INSERT HAVING ALIGNMENT GUIDE SURFACES

(71) Applicant: Roger P. Jackson, Prairie Village, KS (US)

(72) Inventors: Roger P. Jackson, Prairie Village, KS (US); James L. Surber, Kansas City, KS (US)

(21) Appl. No.: 18/811,469

(22) Filed: Aug. 21, 2024

Related U.S. Application Data
(63) Continuation of application No. 17/897,990, filed on Aug. 29, 2022, now Pat. No. 12,070,249.
Application No. 17/897,990 is a continuation of application No. 17/473,580, filed on Sep. 13, 2021, now Pat. No. 11,426,208.
Application No. 17/473,580 is a continuation of application No. 16/685,695, filed on Nov. 15, 2019, now Pat. No. 11,116,547.
Application No. 16/685,695 is a continuation of application No. 16/118,079, filed on Aug. 30, 2018, now Pat. No. 10,478,229.
Application No. 16/118,079 is a continuation of application No. 14/730,981, filed on Jun. 4, 2015, now Pat. No. 10,064,658.
(60) Provisional application No. 62/007,623, filed on Jun. 4, 2014.

(51) Int. Cl. A61B 17/70; A61B 17/86

(52) U.S. Cl. CPC A61B 17/7037; A61B 17/7032; A61B 17/864

(57) ABSTRACT

A modular pivotal bone anchor assembly includes a receiver having a first open channel for receiving a rod and a central bore with a lower locking region, an upper expansion region, and opposed internal guide surfaces defining a longitudinal alignment channel. The assembly also includes an insert uploadable into the central bore with opposite outer guide surfaces slidably positionable within the longitudinal alignment channel to inhibit rotation between the receiver and insert, and an open ring retainer uploadable into the central bore after the insert and thereafter translatable between the lower locking region and the upper expansion region. The assembly further includes a shank with a shank head uploadable into the central bore with a loading motion that causes the retainer to translate into and expand within the upper expansion region, and then to contract around the shank head and translate back down into the lower locking region, with the shank head being seated against an internal surface of the retainer to secure the shank to the receiver.

### CROSS-REFERENCE TO RELATED APPLICATIONS

This application is a continuation of U.S. application Ser. No. 17/897,990, filed Aug. 29, 2022, now U.S. Pat. No. 12,070,249, which is a continuation of U.S. application Ser. No. 17/473,580, filed Sep. 13, 2021, now U.S. Pat. No. 11,426,208, which is a continuation of U.S. application Ser. No. 16/685,695, filed Nov. 15, 2019, now U.S. Pat. No. 11,116,547, which is a continuation of U.S. application Ser. No. 16/118,079, filed Aug. 30, 2018, now U.S. Pat. No. 10,478,229, which is a continuation of U.S. application Ser. No. 14/730,981, filed Jun. 4, 2015, now U.S. Pat. No. 10,064,658, which claims the benefit of U.S. Provisional Application No. 62/007,623, filed Jun. 4, 2014, each of which is incorporated by reference in its entirety herein, and for all purposes.

### BACKGROUND OF THE INVENTION

Polyaxial bone screws and related anchors of various types have been used for supporting rods and other elongate members in spinal surgery. Some of these bone screws utilize a lower pressure insert to transfer locking forces from a rod or other structure above the insert to a shank below the insert, so as to lock the shank in a fixed angular configuration with respect to a receiver. A problem encountered with the insert is that during assembly of the insert into the receiver and/or during transport and/or during implantation of the anchor into a patient, the insert may undesirably rotate from a preferred alignment and configuration relative to the receiver.

### SUMMARY OF THE INVENTION

A bone anchor assembly, especially a bone screw, includes a shank for implanting into a bone, a receiver for holding the shank and receiving an elongate connecting member such as a rod, a pressure insert with opposed upwardly extending arms and a closure. Preferably, the shank has a spherical head and polyaxially joins with the receiver and is held in the receiver by a retainer that may be joined to the receiver or the shank head. The receiver has upper arms that are spaced and form a channel for receiving the elongate member. The receiver arms include break-off extensions, although in some embodiments no extensions will be included. The closure is advancingly received between the arms and applies locking pressure to the elongate member which in turn applies the pressure to the insert that locks the position of the shank relative to the receiver. The closure can also be configured to apply locking pressure to the insert before independently applying locking pressure to the elongate member.

The insert is preferably uploaded into the receiver, but may be downloaded through the channel in certain embodiments. The insert has a plurality of and particularly four opposed generally vertically aligned corners or shoulders. The receiver has a plurality of and particularly four alignment and positioning guides that form regions for receiving the insert shoulders and that snugly ‘slidingly mate with the shoulders on the insert as the insert is being axially or vertically loaded into the receiver. The insert shoulders and the receiver guides cooperate to properly position the insert in the receiver while preventing the insert from rotating axially relative to the receiver. The insert has upwardly extending arms form an insert channel that then aligns with a similar channel of the receiver to accept the elongate member.

Preferably, the shank is cannulated and is polyaxially moveably in the receiver during positioning and thereafter locked in place.

### BRIEF DESCRIPTION OF THE DRAWINGS

FIG. 1 is an exploded view of a bone screw including a shank, a receiver, a retainer, a pressure insert and a closure, shown in conjunction with a rod.

FIG. 2 is a cross sectional view of the bone screw taken along line 2-2 of FIG. 3.

FIG. 3 is a top plan view of the bone screw.

FIG. 4 is a side elevational view of the receiver.

FIG. 5 is a cross sectional view of the receiver, taken along line 5-5 of FIG. 6.

FIG. 6 is a bottom plan view of the receiver.

FIG. 7 is a top plan view of the receiver.

FIG. 8 is a perspective view of the retainer.

FIG. 9 is a top plan view of the retainer.

FIG. 10 is a side elevational view of the retainer.

FIG. 11 is a cross sectional view of the retainer, taken along line 11-11 of FIG. 9.

FIG. 12 is a perspective view of the insert.

FIG. 13 is a top plan view of the insert.

FIG. 14 is a side elevational view of the insert.

FIG. 15 is a bottom plan view of the insert.

FIG. 16 is a perspective view of the receiver, insert and retainer with portions of the retainer cut away to show cooperation of the parts at a stage whereat the insert is being positioned in the receiver.

FIG. 17 is a perspective view of the receiver, insert and retainer with portions of the retainer cut away to show cooperation of the parts at a stage whereat the insert is in an upper most position relative to the receiver.

FIG. 18 is a partial top plan view of the combined receiver and insert with portions of the retainer broken away to illustrate mating of the insert and retainer.

FIG. 19 is a cross section of a side elevational view of the receiver, taken along line 19-19 of FIG. 7 showing the receiver before assembly of the bone anchor.

FIG. 20 is a cross section of the receiver, as in FIG. 19, showing a first stage of the positioning of the insert in the receiver.

FIG. 21 is a cross section of the receiver as in FIG. 20 showing a second stage of the positioning of the retainer in the receiver.

FIG. 22 is a cross section of the receiver as in FIG. 20 showing a third stage of the positioning of the shank head in the receiver with the shank first entering the receiver.

FIG. 23 is a cross section of the receiver as in FIG. 19 showing a fourth stage of the positioning of the insert in the receiver with the shank head having passed through the retainer and being captured in the receiver.

FIG. 24 is a cross section of the receiver as in FIG. 19 showing view of a fifth stage of the positioning of the insert in the receiver with a rod and closure added and the closure applying pressure only to insert.

FIG. 25 is a cross section of the receiver as in FIG. 19 showing a sixth stage of the positioning of the insert in the receiver showing the closure pushing the rod downwardly into a locked position against the insert.

FIG. 26 is a cross section of the receiver as in FIG. 19 showing a ninth stage of the positioning of the insert in the receiver with the rod secured in place in a channel of the receiver and a head of the closure broken away.

FIG. 27 is a side elevational view of the bone anchor with a rod shown in phantom and with the shank pivoted with respect to the retainer with portions removed to show detail thereof.

FIG. 28 is a perspective view of a second embodiment of a closure usable with the present invention.

FIG. 29 is a cross sectional view of the closure of FIG. 28, taken along line 29-29 of FIG. 28.

FIG. 30 is a side elevational view of a bone anchor assembly with the closure of FIG. 28 and a receiver, rod, pressure insert and retainer with the receiver partially broken away to show detail thereof.

The drawings constitute a part of this specification and include exemplary embodiments of the present invention and illustrate various objects and features thereof.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The reference numeral 1 generally indicates a bone anchor in accordance with the invention. While the illustrated anchor 1 is generally a polyaxial bone screw, it is foreseen that the invention could be utilized with other types of spinal implants that utilize pressure inserts, such as polyaxial bone hooks.

The bone anchor 1 comprises a shank 5, a retainer 6, a pressure insert 7, a receiver 8 and a closure 9 and is used with an elongate member 10.

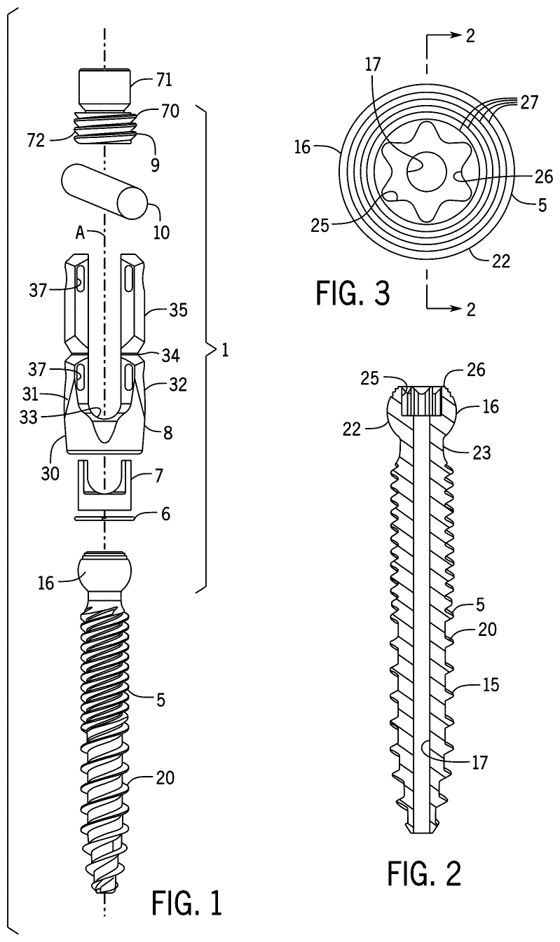

The shank 5 as seen in FIGS. 1 to 3 has a lower portion 15 and an upper portion 16 with an axial bore 17 throughout so as to cannulate the shank 5. The lower portion 15 has a flighting or helical wound thread 20 which is doubled in the upper half for threading into a vertebra of a patient.

The shank upper portion 16 includes a bulbous and partially spherical head 22 that radially extends outward from a neck 23 joining the head 22 to the lower portion 15. Axially centered and extending downward from the top of the head 22 is a tool receiving structure 24 with radially inward extending alternating lobes 25 for receiving and gripping a tool (not shown) used to drive the shank 5 into a bone of a patient. An upper surface 26 of the head 22 has a series of concentric gripping ribs 27 for enhancing frictional contact with the insert 7 which can be made of a somewhat softer metal compared to that of the head.

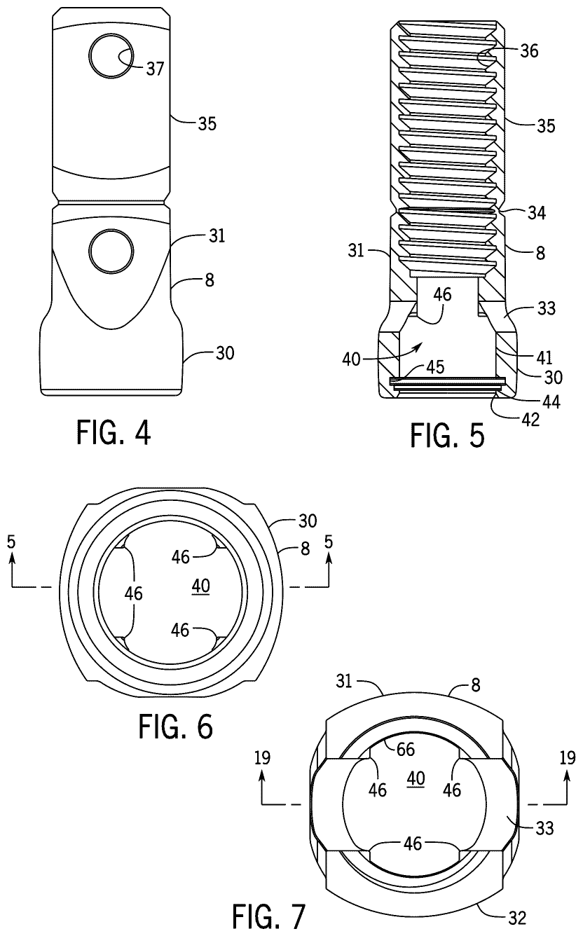

Illustrated in FIGS. 4 to 7 is the receiver 8. The receiver 8 has a lower body 30 and a pair of upstanding spaced arms 31 and 32 forming an elongate member receiving channel 33. In the illustrated embodiment the channel 33 is generally U-shaped, but the shape can be varied to accommodate elongate members of different shapes. Attached by break off junctures 34 to the arms are extensions 35. On facing inner surfaces of the arms are guide and advancement structure which in the illustrated embodiment are helical wound reverse angle thread forms 36, but can be various types of threads, such as a conventional V thread, buttress or square threads or helical flanges. Tool grasping apertures 37 are located on the sides of each arm 31 and 32.

Located in the receiver body 30 is a chamber 40 formed by side walls 41 that opens both into the channel 33 above and to the exterior through a lower opening 42. During assembly of the anchor 1, the chamber 40 receives both the shank head 22 and the insert 7. The lower end of the chamber has a first groove 44 and a larger second groove 45 that are axially aligned with a central Axis A of the receiver, the purpose of which will be discussed later. It is foreseen that the shank can be downloaded into the receiver and not require a retainer, and that the receiver does not have but one groove for the retainer.

Located on the chamber side walls 41 near an upper side thereof are four spaced and radially inward projecting projections or guides 46. The guides 46 cooperate with the insert 7 as discussed below.

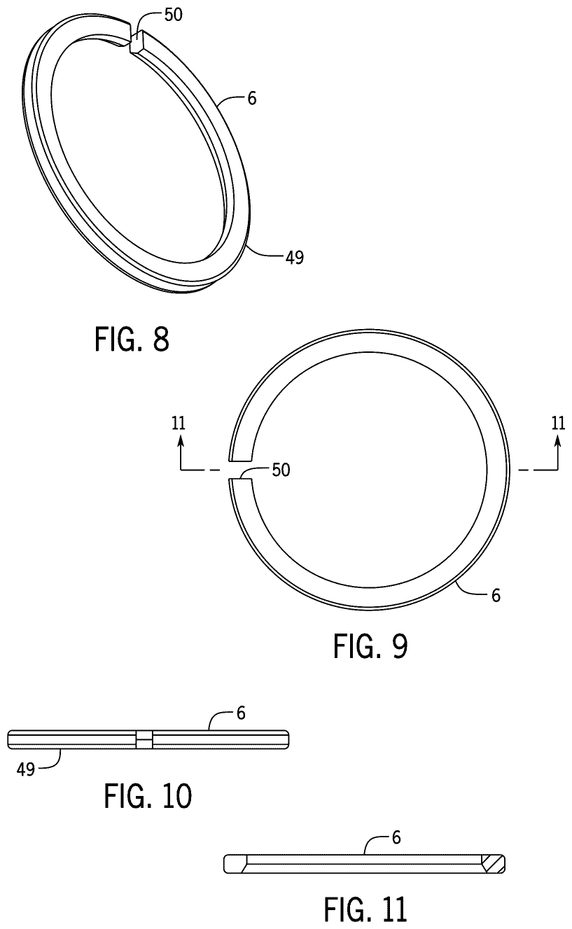

The retainer 6 is shown in FIGS. 8 to 11. The retainer 6 is an open resilient ring 49 with a gap or break 50 to allow contraction and expansion thereof. The ring 49 is compressed and loaded into the receiver. This can occur before or after loading the shank in some embodiments. In the embodiment shown, the ring is loaded first and passes over the shank head 22 during loading of the head 22 into the chamber 40 thereby capturing the shank. During expansion, the ring 49 is received in the larger receiver groove 45 after which the ring 49 is lowered into the smaller groove 44 which is about the same diameter as the ring 49 so that the ring 49 fits snugly therein to prevent repeated expansion. In this manner, the ring 49 holds the shank head 22 in the receiver 8 and allows the shank 5 to pivot relative to the receiver 8 during positioning and before locking.

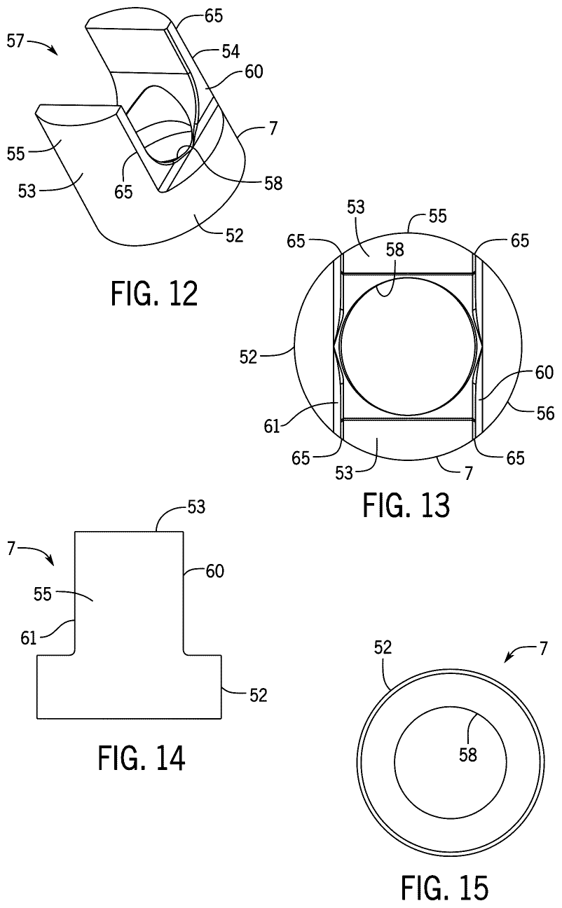

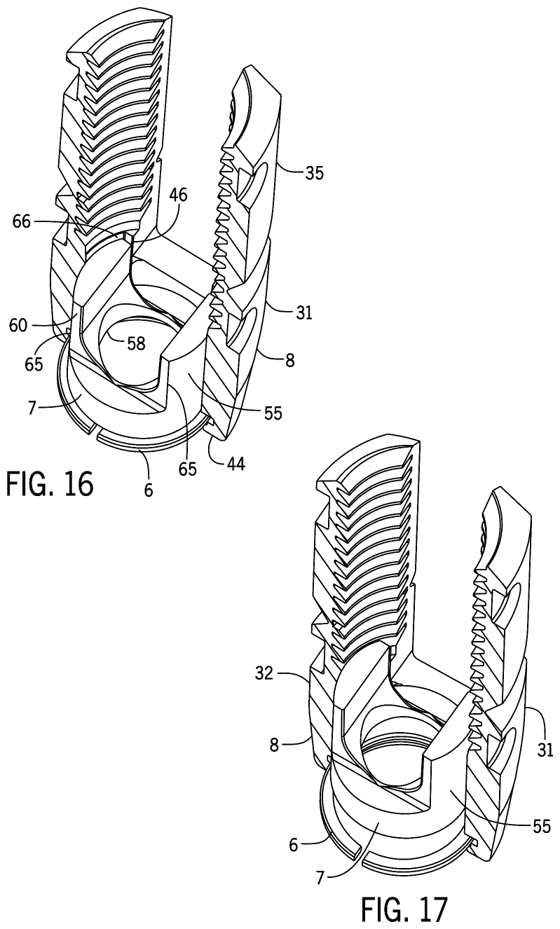

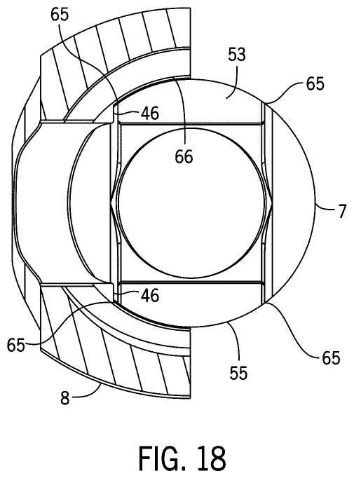

The insert 7 is best seen in FIGS. 12 to 18. The insert 7 includes a lower body 52 with a pair of spaced upstanding arms 53 and 54. The arms 53 and 54 and body 52 have a continuous radially outer surface 55 on each side which are substantially smooth and vertically or axially aligned, but radially spaced from the Axis A. The arms 53 and 54 form a central U-shaped channel 57 therebetween and there is a central axially aligned and centered bore 58.

On either side of the arms 53 and 54 are flat surfaces 60 and 61. At the intersection of the surfaces 60 and 61 with the surfaces 55 and 56 are formed four corners or shoulders 65. The shoulders 65 extend along a length of the insert 7 and are vertically or axially aligned. Each of the shoulders 65 are sized and shaped to vertically slide, but snugly mate with the receiver guides 46. This allows the insert 7 to move vertically during loading into the receiver 8 and during certain positioning required during assembly and implantation of the anchor 1, but prevents the insert 7 from rotating about the axis A relative to the receiver 8. This relationship is perhaps best seen in FIG. 16 wherein the left rear shoulder 65 is seen sliding vertically along the receiver surface 66 and guide 46, but is constrained from axial rotation by the abutment of the shoulder 65 with the guide 46.

The closure 9 is best seen in FIG. 1. The closure 9 has a body 70 with a head 71 that has a tool engagement surface 72 and that breaks from the body 70 at a predetermined torque. Helically wound about the body 70 is a guide and advancement structure 73 which operably mates with the guide and advancement structure 36 on the receiver arms 31 and 32.

The elongate member 10 is for extending between various implants (not shown) in an overall system. The illustrated elongate member 10 is a circular rod, although members of various construction and shape may be utilized.

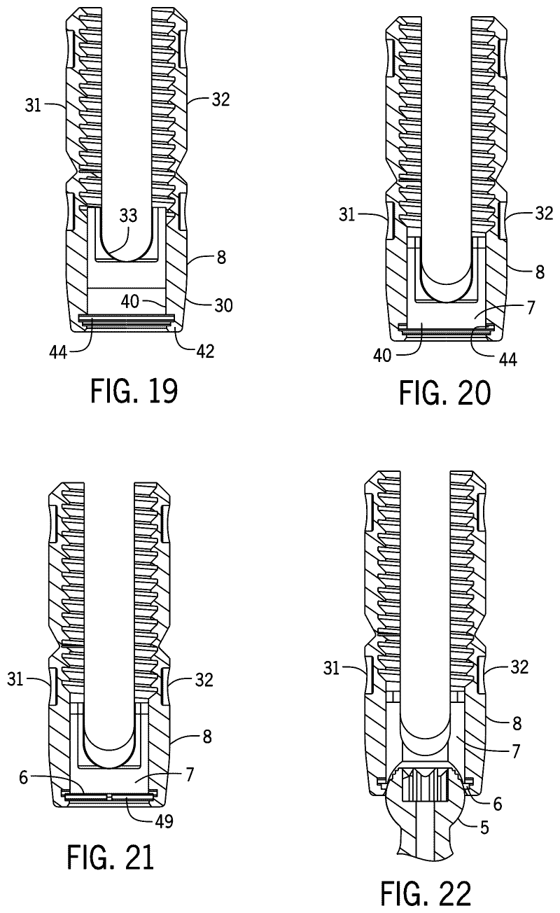

FIGS. 19 to 27 show various stages in the assembly and utilization of the anchor 1 in a snap-on screw embodiment.

The receiver 8 is shown by itself in FIG. 19. In FIG. 20 the pressure insert 7 is uploaded through the opening 42 into the chamber 40. In FIG. 21, the retainer ring 49 is placed in the chamber 40. In FIG. 22, the top of the shank 5 is partially inserted into the chamber 40 and abuts against the retainer 6.

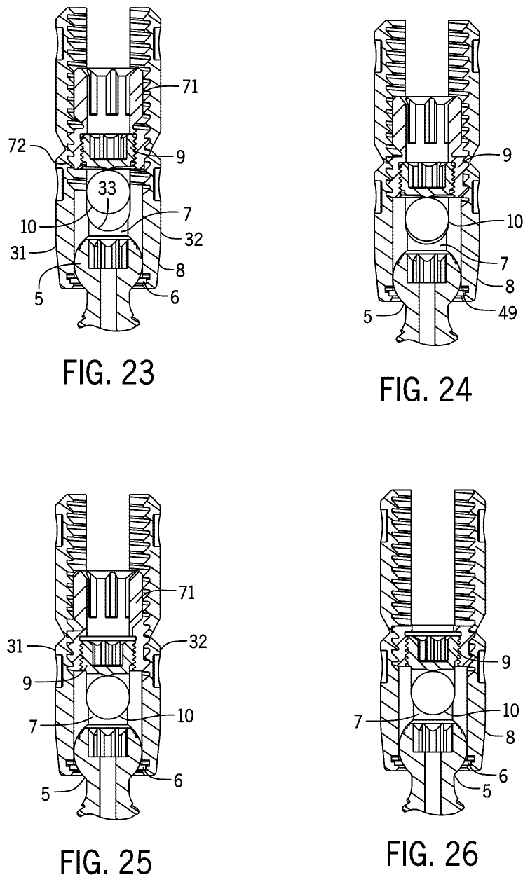

In FIG. 23, the ring 6 has captured the shank head after moving up into the larger receiver groove 45 and coming back down into the smaller groove, while the insert moved upward being aligned and guided by the guides 46. In FIG. 24, the shank head is, again, shown fully captured and the ring 49, which is located around the lower half of the head 22, is shown fully seated in the lower and smaller groove 44. The closure is seen compressing the insert to lock the shank with respect to the receiver before locking the rod.

Shown in FIG. 25, the closure is now also independently locking the rod 10. In FIG. 26, the closure 9 is fully advanced against both the insert and the rod 10 and the head 71 is broken away. The arm extensions 35 can then be broken away (not shown). At this point, the closure 9 applies pressure to the rod 10 and/or pressure insert 7 which applies pressure to the shank 5 thereby locking the shank 5 in a fixed rotational position relative to the axis A of the receiver with the shank implanted in a bone (not shown). Prior to locking the shank 5 is polyaxially rotatable relative to the receiver 8 meaning that the angle of the shank 5 may be varied with respect to the receiver 8 and the axis A.

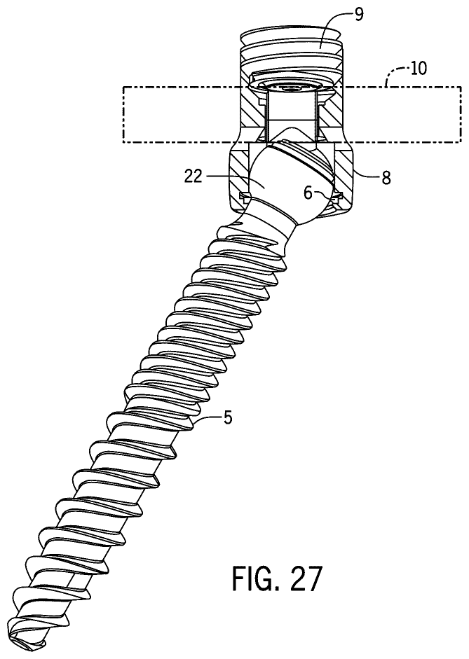

FIG. 27 shows an alternative locked configuration for the polyaxial positioning of the shank 5 relative to the receiver 8.

Once the insert 7 enters the receiver chamber 40, the guides 46 cooperate with the insert shoulder 65 to guide the insert 7 up and down in the receiver 8 while preventing rotation of the insert relative to the receiver 8.

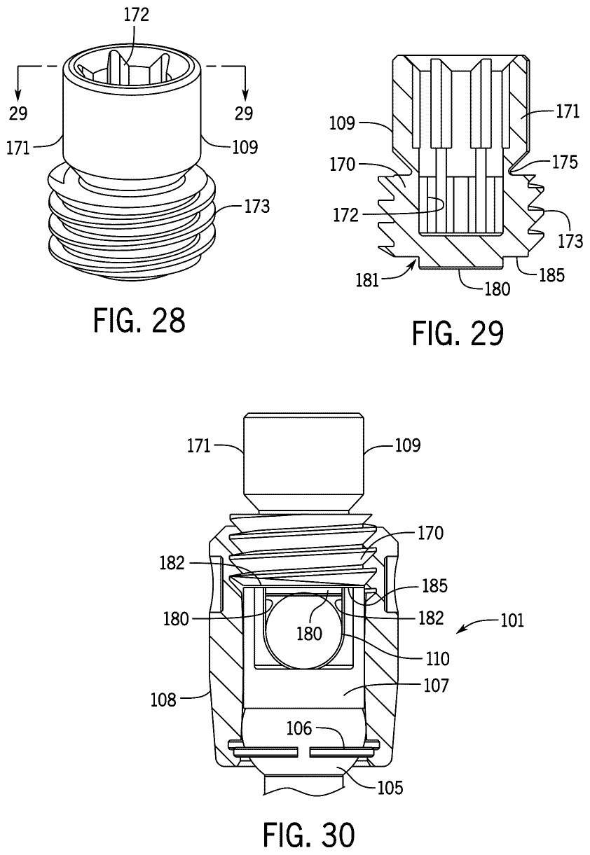

Shown in FIGS. 28 to 30 is an alternative closure for use with the present invention generally identified by the reference numeral 109. It is shown in use in an implant assembly 101 shown in FIG. 30. The closure 109 differs in several aspects from the closure 9. In particular, the closure 9 has an outer ring with a central screw sometimes referred to as a dual innie. The present closure has a unitary body 170 and includes a break off head 171. The body 170 includes outer helical wound reverse angle threads 173 and an internal tool receiving structure 172 for driving the closure 109. The head 171 is attached to the body 170 at a break off neck 175. The head 171 is shown attached to the body 170 in FIG. 30 just prior to breaking away. Importantly, depending from the bottom of the body 170 is a solid circular ring 180 that provides a reduced radius or step down 181.

Seen in FIG. 30 is the closure 109 in a receiver 108 along with a shank 105, a shank retainer 106, a pressure insert 107 and a rod 110. The present insert 107 has two upstanding arms 180 and 181 each with a top surface 182. The arms 180 and 181 are spaced such that the ring 180 on the bottom of the closure 109 passes therebetween on assembly with a slight clearance on each side. A lower surface 185 of the closure body 170 that is radially outward of the ring 180 remains spaced from the insert arms upper surface 182 during assembly and locking. The parts of the assembly 1 and 101 and especially the receiver 8 and 108 and the insert 7 and 107 are preferably constructed of metal that is strong and resists bending or splaying of the arms of either the insert 107 or receiver 108. Preferred material of construction is any grade of titanium and most preferably, cobalt chrome.

It is to be understood that while certain forms of the present invention have been illustrated and described herein, it is not to be limited to the specific forms or arrangement of parts described and shown.

What is claimed is:

1. A modular pivotal bone anchor assembly for securing an elongate rod to a bone of a patient, the pivotal bone anchor assembly comprising:
a shank having an upper capture portion and an anchor portion extending distally from the upper capture portion for fixation to the bone of the patient;
a receiver having a top, a bottom, a central bore extending along a vertical centerline axis of the receiver from the top to the bottom, a base portion defining a lower portion of the central bore communicating with a bottom surface of the base portion through a lower opening, and a first pair of upstanding arms extending upwardly from the base portion to define a first open channel extending transverse to the vertical centerline axis between a front face and an opposite back face of the receiver and configured to receive the elongate rod, the central bore including a lower locking region adjacent the lower opening with a first diameter, an upper expansion region above the lower locking region having a second diameter greater than the first diameter, and at least one pair of opposed internal guide surfaces defining a longitudinal alignment channel communicating with the first open channel;
an insert having a center opening alignable with the vertical centerline axis of the receiver and a second pair of upright arms defining a second open channel extending transverse to the vertical centerline axis, the insert being uploadable into the central bore along the vertical centerline axis prior to the shank with at least one upright arm having opposite outer guide surfaces slidably positionable within the longitudinal alignment channel so as to inhibit rotation between the receiver and the insert and provide guided longitudinal travel therebetween; and
a retainer comprising an open ring having a flat top surface, a bottom surface, a cylindrical external surface, an internal surface, and a vertical slot or gap extending entirely through the open ring from the flat top surface to the bottom surface and from the internal surface to the cylindrical external surface, the retainer being uploadable into the lower portion of the central bore of the receiver after the insert and thereafter being translatable between the upper expansion region and the lower locking region of the central bore,
wherein the upper capture portion of the shank is configured for uploading into the lower portion of the central bore after the insert and the retainer have been positioned within the receiver, with a loading motion of the upper capture portion along the vertical centerline axis of the receiver causing the retainer to first translate into and expand within the upper expansion region, and to then contract around the upper capture portion of the shank and translate back down into the lower locking region, with the upper capture portion of the shank being seated against the internal surface of the retainer to secure the shank to the receiver, and
wherein the insert is configured to engage the upper capture portion of the shank so as to lock the shank with respect to the receiver.

2. The modular pivotal bone anchor assembly of claim 1, wherein the opposite outer guide surfaces of the insert further comprise opposite vertically-extending and parallel flat surfaces facing the front face and the back face of the receiver, respectively.

3. The modular pivotal bone anchor assembly of claim 1, wherein the retainer is non-pivotal with respect to the receiver when positioned within the lower locking region of the central bore.

4. The modular pivotal bone anchor assembly of claim 1, wherein the shank is a polyaxial screw with a threaded shank.

5. The modular pivotal bone anchor assembly of claim 4, wherein the shank has an axial bore centered about a longitudinal axis and extending an entire length of the shank.

6. The modular pivotal bone anchor assembly of claim 1, wherein the upper capture portion of the shank includes a partially spherical upper surface engageable with a concave underside surface of the insert.

7. The modular pivotal bone anchor assembly of claim 6, wherein the insert includes a continuous planar bottom surface extending peripherally and circumferentially entirely around the concave underside surface of the insert.

8. The modular pivotal bone anchor assembly of claim 1, wherein the base portion is formed integrally with the receiver.

9. The modular pivotal bone anchor assembly of claim 1, wherein the longitudinal alignment channel is further defined by a curved interior surface of the central bore of the receiver.

10. The modular pivotal bone anchor assembly of claim 9, wherein an outer surface of the at least one upright arm of the insert further includes a curved exterior surface complementary with the curved interior surface of the longitudinal alignment channel.

11. The modular pivotal bone anchor assembly of claim 1, wherein the upright arms of the insert include parallel planar top surfaces.

12. The modular pivotal bone anchor assembly of claim 11, wherein the insert further includes an upward facing planar surface below the parallel planar top surfaces and extending perpendicular to the vertical centerline axis to an outer edge of the insert.

13. The modular pivotal bone anchor assembly of claim 1, wherein the receiver further includes breakoff extensions extending upwardly from the first pair of upstanding arms of the receiver.

14. A method of assembling a modular pivotal bone anchor assembly configured for securing an elongate rod to a bone of a patient, the method comprising:
uploading an insert into a central bore of a receiver,
the receiver comprising a top, a bottom, the central bore extending along a vertical centerline axis of the receiver from the top to the bottom, a base portion defining a lower portion of the central bore communicating with a bottom surface of the base portion through a lower opening, and a first pair of upstanding arms extending upwardly from the base portion to define a first open channel extending transverse to the vertical centerline axis between a front face and an opposite back face of the receiver and configured to receive the elongate rod, the central bore including a lower locking region adjacent the lower opening with a first diameter, an upper expansion region above the lower locking region having a second diameter greater than the first diameter, and at least one pair of opposed internal guide surfaces defining a longitudinal alignment channel communicating with the first open channel,
the insert comprising a center opening alignable with the vertical centerline axis of the receiver and a second pair of upright arms defining a second open channel extending transverse to the vertical centerline axis, the insert being uploadable into the central bore along the vertical centerline axis prior to the shank with at least one upright arm having opposite outer guide surfaces slidably positionable within the longitudinal alignment channel so as to inhibit rotation between the receiver and the insert and provide guided longitudinal travel therebetween; and
uploading a retainer into the lower portion of central bore after the insert, the retainer comprising an open ring having a flat top surface, a bottom surface, a cylindrical external surface, an internal surface, and a vertical slot or gap extending entirely through the open ring from the flat top surface to the bottom surface and from the internal surface to the cylindrical external surface,
wherein the retainer is translatable between the upper expansion region and the lower locking region of the central bore.

15. The method of claim 14, wherein the retainer is non-pivotal with respect to the receiver when positioned within the lower locking region of the central bore.

16. The method of claim 14, further comprising uploading an upper capture portion of a shank into the lower portion of the central bore after the insert and the retainer have been positioned within the receiver, the shank comprising the upper capture portion and an anchor portion extending distally from the upper capture portion for fixation to the bone of the patient,
wherein a loading motion of the upper capture portion of the shank along the vertical centerline axis of the receiver causes the retainer to first translate into and expand within the upper expansion region, and to then contract around the upper capture portion of the shank and translate back down into the lower locking region, with the upper capture portion of the shank being seated against the internal surface of the retainer to secure the shank to the receiver.

17. The method of claim 16, wherein the insert is configured to engage the upper capture portion of the shank so as to lock the shank with respect to the receiver.

18. The method of claim 17, wherein the upper capture portion of the shank includes a partially spherical upper surface engageable with a concave underside surface of the insert.
